(19) United States
(12) Patent Application Publication
(10) Pub. No.: US 2013/0096275 A1
(43) Pub. Date: Apr. 18, 2013

(54) VANCOMYCIN B HYDROCHLORIDE CRYSTALLINE FORM 1

(71) Applicant: Abbott Laboratories, Abbott Park, IL (US)

(72) Inventors: Stephen S. Ulrey, Lake Bluff, IL (US); Michael B. DeMarco, Wadsworth, IL (US); Li Sun, Grayslake, IL (US); Mark W. Turbergen, Mount Prospect, IL (US); Thomas J. Paulus, Grayslake, IL (US)

(73) Assignee: Abbott Laboratories, Abbott Park, IL (US)

(21) Appl. No.: 13/693,831

(22) Filed: Dec. 4, 2012

Related U.S. Application Data
(63) Continuation of application No. 13/412,886, filed on Mar. 6, 2012, now abandoned.
Application No. 13/412,886 is a continuation of application No. 12/635,494, filed on Dec. 10, 2009, now abandoned.
Application No. 12/635,494 is a continuation of application No. 11/956,413, filed on Dec. 14, 2007, now abandoned.
(60) Provisional application No. 60/882,656, filed on Dec. 29, 2006.

(51) Int. Cl. C07K 1/30; C07K 9/00

(52) U.S. Cl. CPC C07K 1/306; C07K 9/008
USPC 530/322

(57) ABSTRACT

Vancomycin B Hydrochloride Crystalline Form 1, compositions containing it and methods of prevention or treatment of bacterial infections using it are disclosed.

### RELATED APPLICATION

This application claims priority to U.S. patent application Ser. No. 60/882,656, filed Dec. 29, 2006 and is incorporated herein by reference in its entirety.

### FIELD OF THE INVENTION

This invention pertains to Vancomycin B Hydrochloride Crystalline Form 1, ways to make it, compositions containing it and methods of treatment of diseases using it.

### BACKGROUND OF THE INVENTION

Vancomycin B hydrochloride is a salt of Vancomycin B (C₆₆H₇₅Cl₂N₉O₂₄) that is produced by the actinomycete Amycolaptosis orientalis (formerly Streptomyces orientalis) found in Indonesian and Indian soil.

Because the relationship between different crystalline forms of Vancomycin B Hydrochloride may provide guidance for further development, there is an existing need in the chemical and therapeutic arts for identification of different crystalline forms of Vancomycin B Hydrochloride and ways of reproducibly making it.

### SUMMARY OF THE INVENTION

One embodiment of this invention pertains to Vancomycin B Hydrochloride Crystalline Form 1.

Still another embodiment of this invention pertains to Vancomycin B Hydrochloride Crystalline Form 1 having substantial crystalline purity.

Still another embodiment pertains to compositions made with or comprising an excipient and Vancomycin B Hydrochloride Crystalline Form 1.

Still another embodiment pertains to methods for treating bacterial infection in a human comprising administering thereto a therapeutically effective amount of Vancomycin B Hydrochloride Crystalline Form 1.

Still another embodiment pertains to a process for making Vancomycin B Hydrochloride Crystalline Form 1 comprising:

providing a mixture comprising vancomycin and solvent, wherein the Vancomycin B hydrochloride is completely dissolved in the solvent;

causing Vancomycin B Hydrochloride Crystalline Form 1 to exist in the mixture; and isolating the Vancomycin B Hydrochloride Crystalline Form 1.

Still another embodiment pertains to Vancomycin B Hydrochloride Crystalline Form 1 prepared as described in the preceding process.

Still another embodiment pertains to a process for making Vancomycin B Hydrochloride Crystalline Form 1 comprising:

providing a mixture comprising Vancomycin B hydrochloride and one volume of water, wherein the Vancomycin B hydrochloride is completely soluble in the water;

causing Vancomycin B Hydrochloride Crystalline Form 1 to exist in the mixture by adding 1.5 volumes of acetone to the mixture; and isolating the Vancomycin B Hydrochloride Crystalline Form 1.

Still another embodiment pertains to Vancomycin B Hydrochloride Crystalline Form 1 prepared as described in the preceding process.

In a process for making Vancomycin B Hydrochloride Crystalline Form 1 by fermentation and subsequent crystallization or recrystallization of Vancomycin B to the Vancomycin B Hydrochloride Crystalline Form 1, still another embodiment comprises direct formation of Vancomycin B Hydrochloride Crystalline Form 1 from a solid having therewith at least one residual solvent selected from the group consisting of water, acetone and a mixture thereof.

Still another embodiment pertains to Vancomycin B Hydrochloride Crystalline Form 1 prepared as described in the preceding process.

### DETAILED DESCRIPTION OF THE INVENTION

This invention pertains to discovery of Vancomycin B Hydrochloride Crystalline Form 1, ways to make it having substantial crystalline purity, ways to characterize it, compositions containing it and methods of treatment of bacterial infections using it.

The term “amorphous,” as used herein, means a supercooled liquid or a viscous liquid which looks like a solid but does not have a regularly repeating arrangement of molecules that is maintained over a long range and does not have a melting point but rather softens or flows above its glass transition temperature.

The term “anti-solvent,” as used herein, means a solvent in which a compound is substantially insoluble.

The term “Vancomycin B Hydrochloride Crystalline Form 1,” as used herein, means a particular crystalline form of Vancomycin B that is the most thermodynamically stable crystalline form at 25° C.

The term “chemical purity,” as used herein, means percentage of a particular compound in a sample. A sample of Vancomycin B Hydrochloride Crystalline Form 1 may contain, for example, Vancomycin B Hydrochloride Crystalline Form 1, water and acetone.

The term “crystalline,” as used herein, means having a regularly repeating arrangement of molecules or external face planes.

The term “crystalline purity,” as used herein, means percentage of Vancomycin B Hydrochloride Crystalline Form 1 in a sample that may contain amorphous Vancomycin B, at least one crystalline form of Vancomycin B other than Vancomycin B Hydrochloride Crystalline Form 1 or mixtures thereof.

The term “isolating” as used herein, means separating a compound from a solvent, anti-solvent, or a mixture of solvent and anti-solvent to provide a solid, semisolid or syrup. This is typically accomplished by means such as centrifugation, filtration with or without vacuum, filtration under positive pressure, distillation, evaporation or a combination thereof. Isolating may or may not be accompanied by purifying during which the chemical, chiral or chemical and chiral purity of the isolate is increased. Purifying is typically conducted by means such as crystallization, distillation, extraction, filtration through acidic, basic or neutral alumina, filtration through acidic, basic or neutral charcoal, column chromatography on a column packed with a chiral stationary phase, filtration through a porous paper, plastic or glass barrier, column chromatography on silica gel, ion exchange chromatography, recrystallization, normal-phase high performance liquid chromatography, reverse-phase high performance liquid chromatography, trituration and the like.

The term “miscible,” as used herein, means capable of combining without separation of phases.

The term “solvate,” as used herein, means having on a surface, in a lattice or on a surface and in a lattice, a solvent such as water, acetic acid, acetone, acetonitrile, benzene, chloroform, carbon tetrachloride, dichloromethane, dimethylsulfoxide, 1,4-dioxane, ethanol, ethyl acetate, butanol, tert-butanol, N,N-dimethylacetamide, N,N-dimethylformamide, formamide, formic acid, heptane, hexane, isopropanol, methanol, methyl ethyl ketone, 1-methyl-2-pyrrolidinone, mesitylene, nitromethane, polyethylene glycol, propanol, 2-propanone, pyridine, tetrahydrofuran, toluene, xylene, mixtures thereof and the like. A specific example of a solvate is a hydrate, wherein the solvent on the surface, in the lattice or on the surface and in the lattice, is water. Hydrates may or may not have solvents other than water on the surface, in the lattice or on the surface and in the lattice of a substance.

The term “substantial chemical purity,” as used herein, means about 95% chemical purity, preferably about 97% chemical purity, more preferably about 98% chemical purity, and most preferably about 100% chemical purity.

The term “substantial crystalline purity,” as used herein, means at least about 95% crystalline purity, preferably about 97% crystalline purity, more preferably about 99% crystalline purity, and most preferably about 99.9% crystalline purity.

The term “supersaturated,” as used herein, means having a compound in a solvent in which it is completely dissolved at a certain temperature but at which the solubility of the compound in the solvent at that certain temperature is exceeded.

Unless stated otherwise, percentages stated throughout this specification are weight/weight (w/w) percentages.

Mixtures comprising Vancomycin B Hydrochloride Crystalline Form 1 and solvent may or may not have chemical and diastereomeric impurities, which, if present, may be completely soluble, partially soluble or essentially insoluble in the solvent. The level of chemical or diastereomeric impurity in the mixture may be lowered before or during isolation of Vancomycin B Hydrochloride Crystalline Form 1 by means such as distillation, extraction, filtration through acidic, basic or neutral alumina, filtration through acidic, basic or neutral charcoal, column chromatography on a column packed with a chiral stationary phase, filtration through a porous paper, plastic or glass barrier, column chromatography on silica gel, ion exchange chromatography, recrystallization, normal-phase high performance liquid chromatography, reverse-phase high performance liquid chromatography, trituration and the like.

Causing Vancomycin B Hydrochloride Crystalline Form 1 to exist in a mixture comprising Vancomycin B Hydrochloride and solvent, wherein the Vancomycin B Hydrochloride is completely dissolved in the solvent, is nucleation. In a preferred embodiment for the practice of this invention, nucleation of Vancomycin B Hydrochloride is made to occur in a solvent which is supersaturated with Vancomycin B Hydrochloride.

Mixtures of Vancomycin B Hydrochloride Crystalline Form 1 and solvent, wherein the Vancomycin B Hydrochloride Crystalline Form 1 is completely dissolved in the solvent may be prepared from a crystalline Vancomycin B, amorphous Vancomycin B or a mixture thereof, wherein the crystalline Vancomycin B Crystall and amorphous Vancomycin B may or may not be substantially chemically, diastereomerically or chemically and diastereomerically pure.

For the practice of this invention, nucleation may be made to occur in a solution by techniques that are well-known to those skilled in the art such as, for example, solvent removal, temperature change, solvent-miscible anti-solvent addition, solvent-immiscible anti-solvent addition, seed crystal addition of Vancomycin B Hydrochloride Crystalline Form 1, chafing or scratching the interior of the container, preferably a glass container with a glass rod or a glass bead or beads, or by a combination thereof.

It is meant to be understood that, because many solvents and anti-solvents contain impurities, the level of impurities in solvents and anti-solvents for the practice of this invention, if present, are at a low enough concentration that they do not interfere with the intended use of the solvent in which they are present.

The following examples are presented to provide what is believed to be the most useful and readily understood description of procedures and conceptual aspects of this invention.

### Example 1

A mixture of Vancomycin B hydrochloride in 1 volume of water, in which the Vancomycin B hydrochloride was completely soluble in the water, at 25° C. was treated with 1.5 volumes of acetone (relative to the water). The mixture was stirred for 24-36 hours to precipitate Vancomycin B Hydrochloride Crystalline Form 1. Additional acetone (3 volumes relative to the water) was added at a rate of 150 mL/minute to precipitate additional product. The mixture was centrifuged and filtered; and the filtrant was air dried.

Vancomycin B Hydrochloride Crystalline Form 1 may be characterized by powder diffraction data, single crystal data, or a combination thereof.

A sample of Vancomycin B Hydrochloride Crystalline Form 1 for powder diffraction analysis was applied as a thin layer, with no prior grinding, to the analysis well of a Scintag×2 Diffraction Pattern System having the following parameters: x-ray source: Cu—Kα; range: 2.00°-40.00° 2θ; scan rate: 1.00 degree per minute; step size: 0.02°; temperature: about 25° C.; wavelength: 1.54178 Å (Cu—Kα).

It is meant to be understood that peak heights may vary and will be dependent on variables such as the temperature, size of crystal size or morphology, sample preparation, or sample height in the analysis well of the Scintag×2 Diffraction Pattern System.

It is also meant to be understood that peak positions may vary when measured with different radiation sources. For example, Cu—Kα₁, Mo—Kα, Co—Kα and Fe—Kα radiation, having wavelengths of 1.54060 Å, 0.7107 Å, 1.7902 Å and 1.9373 Å, respectively, may provide peak positions that differ from those measured with Cu—Kα radiation.

The term “about” preceding a series of peak positions is meant to include all of the peak positions of the group which it precedes.

The term “about” preceding a series of peak positions means that all of the peaks of the group which it precedes are reported in terms of angular positions with a variability of ±0.1°.

Vancomycin B Hydrochloride Crystalline Form 1 is an antibacterial and is useful for prevention or treatment of staphylococci or spirochetes bacterial indections.

Compositions made with or comprising Vancomycin B Hydrochloride Crystalline Form 1 may be administered, for example, bucally, ophthalmically, orally, osmotically, parenterally (intramuscularly, intrasternally, intravenously, subcutaneously), rectally, topically, transdermally, or vaginally. Ophthalmically administered dosage forms may be administered as, for example, elixirs, emulsions, microemulsions, oinments, solutions, suspensions, or syrups.

Orally administered solid dosage forms may be administered as, for example, capsules, dragees, emulsions, granules, pills, powders, solutions, suspensions, tablets, microemulsions, elixirs, syrups, or powders for reconstitution. Osmotically and topically administered dosage forms may be administered as, for example, creams, gels, inhalants, lotions, ointments, pastes, or powders. Parenterally administered dosage forms may be administered, as, for example, aqueous or oleaginous suspensions. Rectally and vaginally dosage forms may be administered, for example, as creams, gels, lotions, ointments, or pastes.

The therapeutically acceptable amount of Vancomycin B Hydrochloride Crystalline Form 1 depends on recipient of treatment, disorder being treated and severity thereof, composition containing it, time of administration, route of administration, duration of treatment, its potency, its rate of clearance and whether or not another drug is co-administered. The amount of Vancomycin B Hydrochloride Crystalline Form 1 used to make a composition to be administered daily to a patient in a single dose or in divided doses is from about 0.03 to about 200 mg/kg body weight. Single dose compositions contain these amounts or a combination of submultiples thereof.

Vancomycin B Hydrochloride Crystalline Form 1 may be administered with or without an excipient and with or without at least one additional chemotherapeutic agent. Excipients include, for example, encapsulating materials or additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, processing aids, releasing agents, shell excipients, sterilizing agents, sweeteners, solubilizers, wetting agents and mixtures thereof.

Excipients for preparation of compositions made with or comprising Vancomycin B Hydrochloride Crystalline Form 1 to be administered orally in solid dosage forms include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, FD&C Yellow No. 6, fractionated coconut oil, gelatin such as Gelatin Type 195, germ oil, glucose, glycerol, glycerin, groundnut oil, hydroxypropylmethyl celluose, isopropanol, isotonic saline, lactose, lecithin, magnesium hydroxide, magnesium stearate, malt, mannitol, monoglycerides, olive oil, peanut oil, phosphatidylcholine, polyethylene glycol 600, propylene glycol, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, Sorbitol Special (sorbitol, sorbitol anhydrides and mannitol), soybean oil, stearic acids, stearyl fumarate, sucrose, surfactants, talc, tragacanth, tetrahydrofurfuryl alcohol, titanium dioxide, triglycerides, water, and mixtures thereof.

Excipients for preparation of compositions made with Vancomycin B Hydrochloride Crystalline Form 1 to be administered ophthalmically or orally in liquid dosage forms include, for example, 1,3-butylene glycol, castor oil, corn oil, cottonseed oil, ethanol, fatty acid esters of sorbitan, germ oil, groundnut oil, glycerol, isopropanol, olive oil, polyethylene glycols, propylene glycol, sesame oil, water and mixtures thereof. Excipients for preparation of compositions made with

Vancomycin B Hydrochloride Crystalline Form 1 to be administered osmotically include, for example, chlorofluorohydrocarbons, ethanol, water and mixtures thereof. Excipients for preparation of compositions made with Vancomycin B Hydrochloride Crystalline Form 1 to be administered parenterally include, for example, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water and mixtures thereof. Excipients for preparation of compositions made with or comprising Vancomycin B Hydrochloride Crystalline Form 1 to be administered rectally or vaginally include, for example, cocoa butter, polyethylene glycol, wax and mixtures thereof

The foregoing is meant to be illustrative of the invention and not meant to limit it to disclosed embodiments. Variations and changes obvious to one skilled in the art are intended to be within the scope and nature of the invention as defined in the appended claims.

We claim:

1. A process for making Vancomycin B Hydrochloride Crystalline Form 1 comprising:
providing a mixture comprising Vancomycin B hydrochloride and one volume of water, wherein the vancomycin B hydrochloride is completely soluble in the water;
causing Vancomycin B Hydrochloride Crystalline Form 1 to exist in the mixture by adding 1.5 volumes of acetone to the mixture; and
isolating the Vancomycin B Hydrochloride Crystalline Form 1.
